(19) United States
(12) Patent Application Publication
(10) Pub. No.: US 2019/0042697 A1
(43) Pub. Date: Feb. 7, 2019

(54) COMPUTER-IMPLEMENTED METHODS FOR AUTOMATED ANALYSIS AND PRIORITIZATION OF VARIANTS IN DATASETS

(71) Applicant: THE REGENTS OF THE UNIVERSITY OF MICHIGAN, Ann Arbor, MI (US)

(72) Inventors: Mark J. Kiel, Ann Arbor, MI (US); Kojo Elenitoba-Johnson, Ann Arbor, MI (US); Megan Lim, Ann Arbor, MI (US)

(21) Appl. No.: 16/155,046

(22) Filed: Oct. 9, 2018

Related U.S. Application Data
(63) Continuation-in-part of application No. 14/590,427, filed on Jan. 6, 2015, now abandoned.
(60) Provisional application No. 61/924,450, filed on Jan. 7, 2014.

(51) Int. Cl. G06F 19/24; G06F 19/28; G16H 50/20; G06F 19/18

(52) U.S. Cl. CPC G06F 19/24; G06F 19/18; G16H 50/20; G06F 19/28

(57) ABSTRACT

Computer-implemented methods for automating identification and prioritization of genomic variants are disclosed. Such methods employ a rule set to analyze information regarding statistical frequency of variants in a dataset and metrics indicating biological relatedness to generate a priority-score indicative of the relevance of each variant in the dataset. The methods perform both variant frequency normalization and universal pairwise variant comparisons across the datasets to automatically calculate the likelihood that each variant is significant to a disease or other biological phenomenon under study. Priority-scores are calculated for the variants based upon such pairwise comparisons, and the results are organized into a priority ranking, which may be used to categorize the results into data subsets for display to a user.

This application is a continuation-in-part of U.S. patent application Ser. No. 14/590,427 (filed Jan. 6, 2015), which claims the benefit under 35 U.S.C. § 119(e) of U.S. Provisional Patent Application Ser. No. 61/924,450 (filed Jan. 7, 2014), each of which is hereby expressly incorporated by reference herein.

### TECHNICAL FIELD

The present disclosure relates to computer-implemented methods and algorithms for automatically analyzing genomic variants and, in particular, for automatically identifying and prioritizing genomic variants of significance from datasets containing genome sequence data.

### BACKGROUND

Genes are the functional unit of human biology and are encoded in DNA sequence. Collectively, the sequence of all genes from any individual is called a genome. Any smaller component or components of the genome (e.g., chromosomal regions, entire panels of genes or chromosomal regions, entire sets of coding regions of a given genome or genomes, etc.) are also referred to as genome DNA. Recent technological advances have allowed researchers to discover the sequence of genome DNA, which is revolutionizing the process of discovery in biomedical research and paving the way for the implementation of personalized medicine by fostering individualized diagnosis and treatment of diseases as well as better understanding of the origin of human diversity.

In humans, 99.9% of genome sequence identity is shared. Variations at sites representing the remaining 0.1% are responsible for the phenotypic variation between individuals including the differences in risk for various diseases such as cancers, infectious diseases, autoimmune disorders, etc., or otherwise in how individuals look or behave phenotypically. For an individual (or a tissue sample taken from an individual), the sequencing of genome DNA identifies hundreds of thousands of genetic changes or variants when compared to a standardized and universally accepted reference genome sequence. There is a potential for any one of these genomic variants to play an important role in conferring disease, informing the treatment of a medical condition, or allowing the discovery of biological information. However, the vast majority of variants are common variants that are present at non-zero frequencies among healthy individuals. As such, these common variants represent random chance DNA changes that have occurred within individuals at some point in their evolutionary history and have been passed down through subsequent generations. Consequently, the vast majority of variants do not have any meaningful role in human disease. Among the remaining variants, most of them are inert as they do not lead to any changes in biological function either because of their location within the gene or because of the DNA changes that have occurred. Finally, while some of the remaining variants do cause certain biological changes to occur, these variants are nevertheless irrelevant or unimportant to the biological process or phenomenon being investigated.

The ultimate goal of genome sequence interpretation is to categorize the hundreds of thousands of genomic variants within any given genome sequence dataset and to identify candidates for meaningful variants for use in clinical decision making such as diagnosis and treatment, for use in further scientific investigations, or otherwise to understand the genetic cause of a biological trait. However, because of the massive size of a given genome sequence dataset, a researcher or clinician or other interpreter who obtains the genome sequence dataset faces the challenge of looking through a huge amount of variant information to try to identify the meaningful variants. Some progress has been made in developing techniques or tools for genomic variant analysis, however, to date most lack the ability to perform meaningful, automated variant analysis on the given genome sequence dataset.

The strategy employed by conventional genomic variant analysis tools relies on either eliminating data points that do not meet certain user-defined criteria or highlighting only those variants previously associated with disease states. However, this strategy is dependent on user inputs, and is thus manual in nature and often iterative. Moreover, many data points of potential interest can be either filtered or ignored from consideration based on faulty presuppositions. For example, current variant analysis tools are designed to reduce the variant data size in a way that requires a user to make certain assumptions about the characteristics of candidate variants in the variant data (e.g., which gene will be affected, the frequency in which a variant occurs in a patient population versus a healthy population, whether a variant has been identified in previous studies as being disease-associated, etc.). Once categorized and annotated in this manner, the variant data is then filtered according to some quantitative or qualitative limit set by the user such as filtering the data by limiting the variants to an arbitrary maximum variant frequency, filtering the data by limiting the variants to specific genes, filtering the data by limiting the variants to groups of related genes, etc. Moreover, the ability of current variant analysis tools to accurately identify meaningful variants is also limited by the quality and comprehensiveness of supporting external databases.

Accordingly, the use of current variant analysis tools entails that the user formulate preconceived notions about the characteristics of the candidate meaningful variants and that the user can successfully manipulate the filtering limits through an iterative process of hypothesis generation and testing. However, this cycle of hypothesis generation and testing is often a time-consuming process that does not scale easily or lend itself to automation. Further, this cycle of hypothesis generation and testing can be prone to errors both in terms of false-positive and false-negative results, and may be hindered by the user's own experience and scientific expertise.

### SUMMARY

The features and advantages described in this summary and the following detailed description are not all-inclusive. Many additional features and advantages will be apparent to one of ordinary skill in the art in view of the drawings, specification, and claims hereof. Additionally, other embodiments may omit one or more (or all) of the features and advantages described in this summary.

The present application discloses a method, system, and computer-readable medium storing instructions for automatically identifying and prioritizing variants in a dataset. The method, system, or instructions may perform the following actions: (1) accessing the dataset, wherein the dataset includes genomic sequence data of a target dataset (i.e., an experimental dataset) and a control dataset; (2) calculating a frequency-score for each variant in the target dataset, wherein the frequency-score is based upon statistical frequencies with which the respective variant appears in each of the target dataset and the control dataset; (3) for each pair of variants in the target dataset: (i) performing pairwise comparison between the respective variants of the pair; (ii) calculating a relatedness-score for the pair based upon the pairwise comparison; and (iii) calculating a frequency-corrected relatedness-score for the pair based upon the relatedness-score of the pair and the frequency scores of the respective variants; (4) calculating a control-frequency-score for each variant in the control dataset, wherein the control-frequency-score is based upon statistical frequencies with which the respective variant appears in each of the target dataset and the control dataset; (5) for each control pair of a target variant in the target dataset and a control variant in the target dataset: (i) performing pairwise comparison between the target and control variants of the control pair; (ii) calculating, by the one or more processors, a control-relatedness-score for the control pair based upon the pairwise comparison; and (iii) calculating, by the one or more processors, a control-frequency-corrected relatedness-score for the control pair based upon the control-relatedness-score of the control pair, the frequency score of the target variant, and the control-frequency score of the control variant; (6) calculating a control-frequency-adjusted relatedness-score for each variant in the target dataset, wherein the control-frequency-adjusted relatedness-score for each respective variant is based upon the control-frequency-corrected relatedness-scores of the control pairs in which the respective variant is included in the control pair as the target variant; (7) calculating a normalized frequency-corrected relatedness-score for each pair of variants in the target dataset, wherein the normalized frequency-corrected relatedness-score is associated with one of the variants of the pair and is based upon (i) the frequency-corrected relatedness-score of the pair and (ii) the control-frequency-adjusted relatedness-scores of the one of the variants of the pair; and (8) calculating a priority-score for each variant in the target dataset, wherein the priority-score of each respective variant is based upon the normalized frequency-corrected relatedness-scores associated with the respective variant. The priority-score of each variant may indicate a likelihood that the respective variant contributes to a disease process.

In some embodiments, calculating the frequency-score for each variant in the target dataset may include: (i) calculating a first frequency of the respective variant in the target dataset, (ii) calculating a second frequency of the respective variant in the control dataset, and (iii) calculating the frequency-score based upon a difference between the first frequency and the second frequency. Similarly, calculating the control-frequency-score for each variant in the control dataset may include: (i) calculating a first control-frequency of the respective variant in the target dataset, (ii) calculating a second control-frequency of the respective variant in the control dataset, and (iii) calculating the control-frequency-score based upon a difference between the first control-frequency and the second control-frequency.

In further embodiments, calculating the control-frequency-adjusted relatedness-score for each variant in the target dataset may include summing all the control-frequency-corrected relatedness-scores of the control pairs for which the respective variant is the target variant. Calculating the normalized frequency-corrected relatedness-score for each pair of variants in the target dataset may likewise include dividing the frequency-corrected relatedness-score of the pair by the control-frequency-adjusted relatedness-score of the one of the variants of the pair. In yet further embodiments, calculating the priority-score for each variant may include summing all the normalized frequency-corrected relatedness-scores associated with the respective variant.

Additionally or alternatively, in some embodiments, the one or more processors may be disposed in a plurality of servers and perform at least a portion of the pairwise comparisons by parallel computing in the plurality of servers. Thus, computer-readable instructions may be configured to perform such parallel computing within communicatively connected servers, such as a cloud computing environment.

In further embodiments, performing the pairwise comparison between each pair or control pair of variants may include applying a rule set to calculate a biological relationship between the respective variants. Such biological relationship may comprise one of an intrinsic relationship or an extrinsic relationship. An intrinsic relationship may identify whether two variants are: (i) identical or otherwise at the same genomic position, (ii) in identical domain, or (iii) in identical gene. An extrinsic relationship may identify whether two variants are: (i) within the same functional pathway, (ii) within the same gene family, (ii) in direct or indirect interaction with the same genes, or (iv) have similar gene expression profiles.

### BRIEF DESCRIPTION OF THE DRAWINGS

FIG. 1 is a block diagram of an example system for automatically identifying and prioritizing genomic variants of pathogenic importance from genome sequence datasets.

FIG. 2 is a flow diagram of an example method for automatically identifying and prioritizing genomic variants of pathogenic importance from genome sequence datasets.

FIG. 3 is a diagram illustrating variant frequency normalization on an example experimental dataset.

FIG. 4 is a diagram illustrating pairwise variant comparisons on the example experimental dataset of FIG. 3.

FIG. 5 is a diagram illustrating calculations being applied to the results of FIG. 4.

FIG. 6 is a diagram illustrating calculations being applied to the results of FIG. 5.

FIG. 7 is a block diagram of a computing environment that implements a system and method for automatically identifying and prioritizing genomic variants of pathogenic importance from genome sequence datasets.

The figures depict a preferred embodiment of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

Recent and on-going advances in DNA sequencing technology promise to revolutionize the field of medicine such as the way clinicians understand disease mechanisms, the way disease itself is diagnosed, and the way patients are treated and counseled. Significant changes in the practice of clinical medicine are already occurring as a result of genomic sequencing. Moreover, the potential applications of genome sequencing are likely to extend outside of the field of medicine itself. Specifically, human genome sequencing may play important roles in forensic pathology and law; in social interactions and interpersonal relationships; in psychology and entertainment based on personal information such as genealogy; in data security and cryptology; in military applications and other security operations; and in any research that strives to gain a better understanding of human biology, including but not limited to, human disease, among others. Further, there are many applications of genome sequencing of non-human subjects including organisms associated with the fields of clinical microbiology, livestock husbandry and management, the breeding and sale of domesticated animals, production of botanical specimens in the agriculture and floral industries, etc. Just like the revolution in medicine resulting from the application of human genome sequencing, all of the potential benefits and applications of genome sequencing will require improvements in genome sequencing interpretation and analysis techniques, such as the techniques highlighted by the systems and methods described herein.

Genomic variants denote a single or a grouping of DNA sequences that have undergone changes as referenced against particular sub-populations within particular species due to mutations, recombination/crossover or genetic drift. Examples of the types of genomic variants include single nucleotide polymorphisms (SNPs), copy number variations (CNVs), insertions/deletions (Indels), inversions, translocations, etc.

Genomic variants may be identified through the sequencing of genome DNA. At present, a significant amount of time and effort is required to examine the large number of genomic variants from a genome sequence dataset in order to identify potentially meaningful candidates for analysis and interpretation. Further, once meaningful candidates are isolated, any additional variants of importance must be identified using tedious and error-prone manual data interrogations. As a result, many consequential variants are overlooked and the resulting variant information is often unrefined and incomplete.

However, not all genomic variants are of equal importance. Most genomic variants are common variants that appear in control datasets and play no role in the disease process or biological phenomenon being studied. The likelihood that a variant is important to the disease being studied is directly proportional to the prevalence of that variant in an experimental dataset when compared to the prevalence of that variant in a control dataset. Moreover, most disease processes are genetically multi-factorial having multiple genetic causes but nevertheless all showing a common underlying biological cause. Thus, the variants that are responsible for conferring the disease are not identical but will be instead closely related. Accordingly, for any given disease process or biological phenomenon, multiple variants per individual or group of individuals may be of pathogenic importance and worth further study.

By deprecating common variants and highlighting biological similarities among variants, variant information can be organized and presented in a biologically meaningful manner rapidly and automatically. Described herein are systems and methods that integrate prevalence and other biological and empirical information among genomic variants across experimental and control datasets to automatically identify and prioritize those variants that are most relevant to the disease process or biological phenomenon under study.

When compared to existing techniques, the described systems and methods do not require filtering on variant data, do not require setting limits on the data that is displayed or analyzed, do not rely on foreknowledge of or predictions pertaining to the biological characteristics of meaningful variants, and do not require manual hypothesis testing although one or more of these methods may be used in combination with the described systems and methods of this application. Instead, the described systems and methods analyze variant frequency and other biological and empirical information with respect to all variants in the context of an entire dataset to prioritize potential meaningful candidates. As such, the described systems and methods can produce biologically organized priority-sorted data subsets of variants that are most likely to be of interest to users in a rapid and fully automated process, which is not limited by external database completeness or biological foreknowledge of the users. The end result is a greater utility and increased efficiency of genome sequencing data analysis for diagnosis or enhanced understanding of a disease or biological phenomenon, informed clinical decision making, and ultimately improved patient care.

Thus, while previously existing techniques required subjective judgment of skilled personnel (and thus introduced biases and errors into the process), the techniques described herein enable fully automated variant prioritization. Previously, computer systems were unable to perform such prioritization, instead requiring subjective assessment by humans relying upon their experience and training. By employing the distinct process described herein, however, computer systems are enabled to perform prioritization of genomic variants, which previously required subjective determinations of experienced personnel. The systems and method described herein thus solve the technological problem of enabling automation of genomic variant prioritization by implementing a procedure distinct from the subjective determinations used by humans.

Although variant analysis tools exist, such tools are unable to prioritize variants automatically (i.e., without subjective, user-defined criteria to match for variant filtering). Existing variant analysis tools have various limitations that prevent them from being able to perform automatic variant prioritization, such as: (1) being useful solely for assessment of non-protein coding variants that do not impact protein function and represent a small minority of all total genetic variants, particularly those that contribute to disease (e.g., Haploreg, RegulomeDB, FunSeq, and GWAVA); (2) performing simple variant calling and assessment of protein level changes from nucleotide level change using well-characterized and fundamental laws of genetics (e.g., VEP and ANNOVAR); (3) simply aggregating data for annotation without performing an integration of these values or prioritization of likely pathogenicity (e.g., GEMINI); or (4) utilizing solely evolutionary conservation as a predictor of functional consequence (e.g., VAAST CADD).

Unlike previous techniques, the methods described herein prioritize variants based upon population frequency and biological relatedness. Determining biological relatedness may include identify how close to a significant or consequential region the genetic variant is. Previously existing techniques do not perform such prioritization, nor do they establish a biological relatedness between genetic variants.

For example, programs such as Haploreg only look at mutations in a non-coding region of a gene, of which approximately 1% contribute to human disease. Haploreg invites users to enter a SNP (rsXXXXX), a set of SNPs, and a genomic region, from which it will then provide a readout—leading to user bias. Programs such as Haploreg also prioritize changes in the genome differently than the present method. A program such as Haploreg looks only at non-coding variants and only looks at information intrinsically (i.e., within the same gene and in the absence of any information about variants other than the variant being examined).

Programs that perform variant calling such as VEP and ANNOVAR take a DNA sequence, break it apart, and then reconstruct the DNA and identify deviations from some reference genome sequence. If a change from the reference DNA is identified, such variant calling techniques only identify whether the variant is in the protein coding area. They do not look to the relatedness to any other mutations or coding regions, nor do they assess the likelihood that the variant may cause disease. Such variant calling programs only look at intrinsic information and do not look at the biological relatedness of different genes identified, and therefore cannot develop a relatedness score as described herein since they do not establish any sort of connection between different mutations.

Programs such as GEMINI are simple annotation tools that take in information from external databases with information on genetic variants and provide the user with whatever information was requested. GEMINI and similar programs do not provide any information about other mutations or about relatedness or connections to any other mutations (without human intervention and bias), nor do they set a priority for the mutations identified. As such, these programs also cannot provide a relatedness score as described herein.

Finally, programs such as VAAST or CADD are used in evolutionary biology and only look to genetic changes throughout speciation that may provide some information about functionality. However, these programs do not provide any information about other mutations, nor about relatedness or connections to any other mutations. Moreover, they do not set a priority for the mutations identified. Therefore, these programs also cannot provide a relatedness score as described herein.

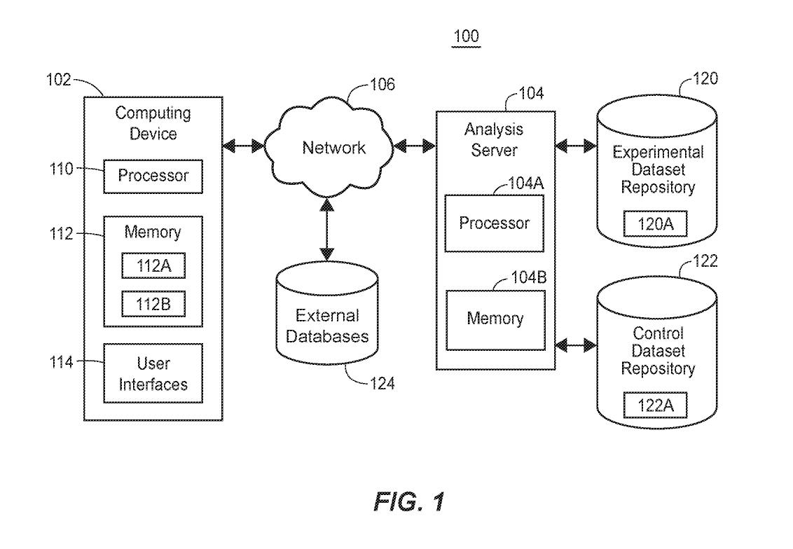

Referring first to FIG. 1, which shows a block diagram of an example system 100 for automatically identifying and prioritizing genomic variants of pathogenic importance from genome sequence datasets. The example system 100 includes a computing device 102 coupled to an analysis server 104 via a communication network 106 that can include wired and/or wireless links. The computing device 102 may be, for example, a laptop computer, a desktop computer, or other devices that can send and receive data over the network 106. In the embodiment shown in FIG. 1, the computing device 102 includes a processor 110, a memory 112, and user interfaces 114 (e.g., a display screen, a touchscreen, a keyboard, etc.). Further, while only one computing device 102 is shown in FIG. 1, the system 100 may include any number of computing devices in other embodiments and/or scenarios.

Generally speaking, a user (e.g. a researcher, a clinician, a health care provider, or any individual with any comprehension of the basic principles of biology) may use the computing device 102 to communicate with the server 104 to perform analysis on one or more given genome sequence datasets. A given genome sequence dataset may be any target dataset (i.e., experimental dataset) obtained from a genome sequencing experiment. For example, the given genome sequence dataset may be obtained from a genome sequencing experiment of a patient population in a clinical trial. As another example, the given genome sequence dataset may be obtained from a genome sequencing experiment of a disease with multiple genetic contributions to disease development (e.g., diabetes mellitus) in a research study. As another example, the genome sequence data may come from an individual patient sample (e.g., a cancer tissue biopsy) along with tissue from the same patient that does not contain cancer cells. As a further example, the genome sequencing data may come from an individual patient sample with a suspected constitutional genetic disorder along with sequencing data from that patient's father, mother and/or other family members. As an additional example, the genome sequencing data may come from an individual without any known medical condition in order to determine the likelihood of later development of a specific disease or other biological phenomenon (such as response to specific medications or prediction of a phenotypic trait such as baldness). Accordingly, the given genome sequence dataset may come from any academic, clinical, or commercial setting where genome sequencing data is produced. Once obtained, the given genome sequence dataset may be stored in the memory 112 as experimental data 112A before being transmitted to the server 104 via the network 106. In some embodiments, the given genome sequence dataset may be sent directly to the server 104 via the network 106.

The analysis server 104 may be a single server or a plurality of servers with distributed processing. The server 104 may be directly coupled to an experimental dataset repository 120 and a control dataset repository 122. In some embodiments, the repository 120 and/or the repository 122 may not be directly coupled to the server 104, but instead may be accessible by the server 104 via a network such as the network 106.

In some embodiments, the server 104 may include a plurality of servers operating in a coordinated manner to perform processing tasks. For example, the server 104 may comprise a cloud processing server group communicatively connected and configured to process large datasets in parallel (i.e., to process parts of datasets on different servers at the same time). Thus, memory or processor demands may be distributed over a plurality of servers, resulting in faster processing of large datasets. In some such embodiments, a control server may coordinate the parallel operations of the other servers, such as by dividing the data and sending parts thereof to the various servers for processing. In further embodiments, the server 104 may perform parallel processing by dividing processing tasks between various cores of a multi-core processor.

The analysis server 104 may receive a given genome sequence dataset or experimental data via the network 106 and store the received data in the experimental dataset repository 120 as experimental dataset 120A. As used herein, the terms “target dataset” and “experimental dataset” are used interchangeably, as are the terms “target dataset repository” and “experimental dataset repository.” In one embodiment, the server 104 receives the experimental data 112A in the memory 112 via the network 106, and stores the received experimental data 112A as the experimental dataset 120A. The server 104 may operate directly on the experimental dataset 120A, or may operate on other data that is generated based on the experimental dataset 120A. For example, the server 104 may convert the data 120A in the repository 120 to a particular format (e.g., for efficient storage), and later utilize the modified data for analysis purposes. Generally speaking, the experimental datasets 112A and/or 120A may include entirely unfiltered experimental data, fully or partially filtered experimental data, subsets of unfiltered or filtered experimental data, or any combination thereof. The analysis server 104 may also receive control data via the network 106 and store the received data in the control dataset repository 122 as control dataset 122A. The control data relates to relevant biological information for individual genomic variants. For example, the relevant biological information may pertain to the prevalence or frequency of individual variants within various disease populations or populations with common phenotypic phenomenon. In some embodiments, the server 104 receives the control data from external databases 124. In other embodiments, the server 104 may receive the control data from a user (e.g., via the computing device 102). In addition, in various embodiments, the control data may be modified according to any desired user specification. Alternatively or additionally, the control data may be received by the computing device 102 and stored in the memory 112 as control data 112B. In general, the analysis server 104 may use zero, one, or multiple control datasets for analysis purposes. Furthermore, control datasets (e.g., the control datasets 112B and/or 122A) can be negative controls (e.g., unaffected by a disease or biological characteristic) or positive controls (e.g., possessing the trait).

With continued reference to FIG. 1, the external databases 124 may include both public and private databases. Examples of publicly accessible databases include the Single Nucleotide Polymorphism Database (dbSNP) provided by the National Center for Biotechnology Information, the HapMap Database provided by the International Haplotype Map Project, the ClinVar database provided by the National Center for Biotechnology Information, etc. In some embodiments, the analysis server 104 and/or the computing device 102 may be configured to gather data from the external databases 124 at regular intervals (e.g., at various times throughout each week, each month, etc.). In other embodiments, data may be automatically requested and sent from the external databases 124 to the server 104 and/or the device 102 through the use of a data refresh executable or script. In this manner, the control dataset 122A in the control dataset repository 122 and/or the control data 112B in the memory 112 can be continuously refreshed as the external databases 124 are updated with new or modified data.

In order to automatically identify and prioritize genomic variants of pathogenic importance, the server 104 may be configured to analyze the relative significance of each genomic variant within both experimental and control datasets. To accomplish this, a processor 104A of the server 104 may execute instructions stored in a memory 1048 of the server 104 to first retrieve the datasets 120A and 122A in the experimental dataset repository 120 and the control dataset repository 122, respectively. The server 104 may then perform variant frequency normalization and universal pairwise variant comparisons across the datasets 120A and 122A to determine a priority ranking, which defines the likelihood that any given variant may contribute to the disease process under study. Once the server 104 determines the priority ranking, the server 104 may generate visualizations for the priority ranking and display the visualizations to the user. For example, the visualizations may be displayed to the user on the user interfaces 114 (e.g., a display screen) of the computing device 102.

In some embodiments, the computing device 102 may be configured to analyze the relative significance of each genomic variant in the experimental and control datasets. In this scenario, the processor 110 may execute instructions stored in the memory 112 to access the data 112A and 1128, and perform variant frequency normalization and universal pairwise variant comparisons on the data 112A and 1128 to determine the priority ranking.

Moreover, as can be seen from the above the discussion, the system 100 drastically shortens the time required for analyzing genomic variants, at least in part by providing a fully automated process to identify and prioritize genomic variants of pathogenic importance. As such, the resource usage or consumption of the system 100 during the analysis process is greatly reduced. For example, the number of processor cycles utilized by the analysis server 104 and/or the computing device 102 from receiving the genomic data to analyzing and prioritizing the data may be greatly reduced by the system 200. Further, the total number of messages or traffic sent over the network 106 during the analysis process is also greatly reduced, thereby increasing efficiencies of the network 106.

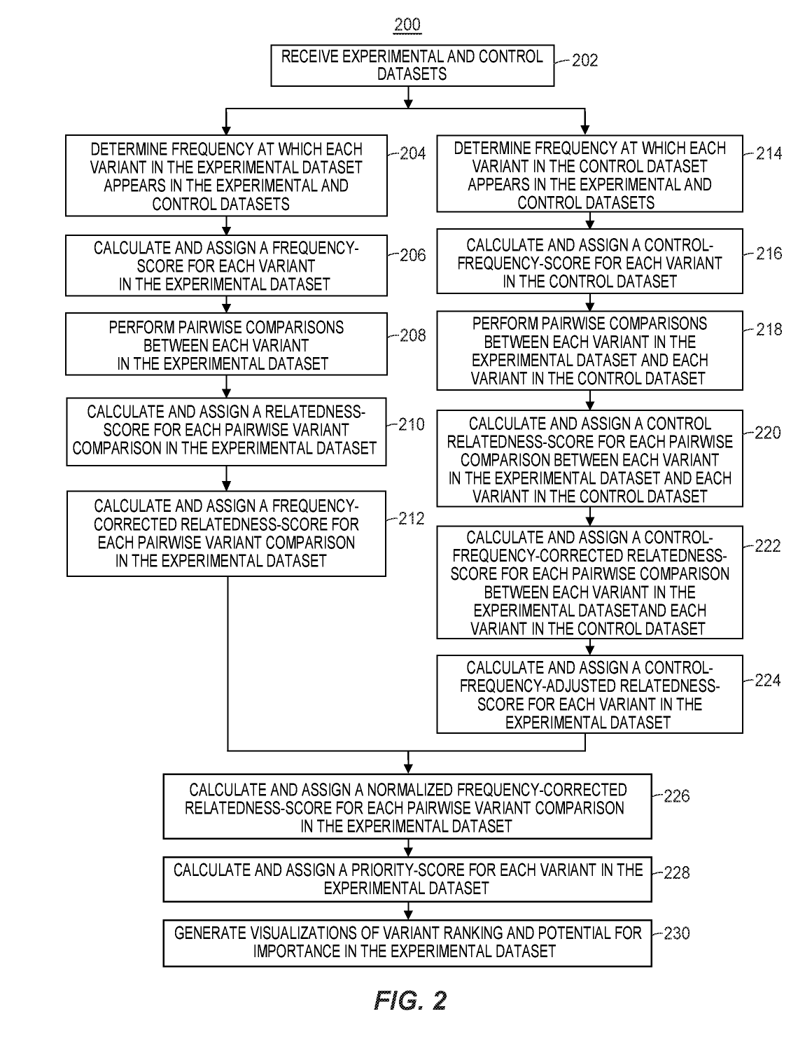

Referring now to FIG. 2, which describes a flow diagram of an example method 200 for automatically identifying and prioritizing genomic variants of pathogenic importance from genome sequence datasets. The method 200 may include one or more blocks, routines or functions in the form of computer executable instructions that are stored in a tangible computer-readable medium (e.g., 104B, 112 of FIG. 1) and executed using a processor (e.g., 104A, 110 of FIG. 1). Generally speaking, the method 200 relates to performing variant frequency normalization and universal pairwise variant comparisons to identify and prioritize which variants are most likely to contribute to the disease or biological phenomenon under study.

The method 200 begins by receiving experimental and control datasets (block 202). For example, with reference to FIG. 1, the method 200 may receive the experiment dataset 120A and the control dataset 122A. The experimental dataset may comprise experimental variant data related to the disease or biological phenomenon being studied and drawn from either an individual or a patient population. The received experimental dataset may include any combination of unfiltered and filtered experimental data. The control dataset may comprise control variant data drawn from an individual or individuals or populations that do not have the disease or trait common to those in the experimental dataset. The method 200 may use zero, one, or multiple control datasets, and thus may receive zero, one, or multiple control datasets. Further, the received control datasets can either be negative controls or positive controls. In some embodiments, the experimental and control datasets may be received as formatted data ready for use in subsequent processing steps. As an example, a received experimental dataset may comprise a file with all the variant information concatenated into a single line defined by various fields indicating chromosome number, chromosomal position, DNA basepair change, amino acid change, etc. In other embodiments, the experimental and control datasets may be received as raw data, and the method 200 may convert the raw data into any desired format, protocol, or information type needed for subsequent processing.

Next, the method 200 proceeds to perform variant frequency normalization and universal pairwise variant comparisons on the received experimental dataset (blocks 204-212) and control dataset (blocks 214-224). While the embodiment of FIG. 2 shows the blocks 204-212 and blocks 214-224 as being in parallel, in other embodiments, these blocks may be in series. For example, the method 200 may execute the blocks 204-212 first before executing the blocks 214-224, or vice versa.

In some embodiments, part of the method 200 may be performed using parallel processing in a multi-core processor or in a multi-processor cloud computing environment, as described elsewhere herein. Such parallel processing may be advantageous in reducing the time required to perform the evaluations and comparisons of the variants, particularly in large datasets. In some such embodiments, the experimental dataset may be processed in parallel with the control dataset, as illustrated in FIG. 2. In further embodiments, groups of variants within each of the experimental and control datasets (or even individual variants) may be processed in parallel to further reduce the time required for analysis and prioritization.

To process the experimental dataset, the method 200 first performs variant frequency normalization to assess the relative importance of each variant in the experimental dataset. The method 200 determines the prevalence or frequency at which each variant in the experimental dataset appears in the experimental dataset and the control dataset (block 204). Deviations from the observed frequency of a given variant within the experimental dataset and the expected frequency of the given variant within the control dataset can be used to qualitatively identify distinct subpopulations of variants that are more likely to be important than others and quantitatively define these subpopulations. For example, if the frequency of a variant in an experimental dataset of individuals with a disease is found to be similar to the frequency of the variant in a control dataset drawn from individuals without the disease, then the variant is unlikely to be meaningful. On the other hand, if a variant is present at a high frequency in the experimental dataset, but at a frequency of near or equal to zero in the control dataset, then the variant is likely to be a meaningful one. In some embodiments, the same calculation can be applied to experimental data from a single genome where the variant frequency in the experimental dataset is either 0 (absent) or 1 (present).

The method 200 then calculates and assigns a frequency-score for each variant in the experimental dataset (block 206). This allows the method 200 to quantitatively measure the relative importance of each variant in the experimental dataset. In an example embodiment, the method 200 takes the frequency values determined for each variant in block 204 and calculates a Pearson's chi-square statistic for each variant. This calculation assesses the probability that the observed frequency of a given variant in the experimental dataset is statistically similar to the expected frequency of the variant in the control dataset. Accordingly, if the observed frequency is close or equal to the expected frequency, then the chi-square statistic will be near or equal to zero (0). This entails that there is a high statistical probability that the variant occurred in the experimental dataset purely by chance (i.e., the variant is a common variant that is unlikely to be meaningful). However, if the observed frequency is much greater (or much less) than the expected frequency, then the chi-square statistic will be a large non-zero value. This entails that there is a low statistical probability that the variant occurred in the experimental dataset purely by chance (i.e., the variant is likely to be meaningful). Thus, by using the chi-square statistic, the method 200 can quantitatively assess the meaningfulness of each variant relative to one another in the experimental dataset. It should be noted, however, that in some embodiments, the method 200 may use a different type of statistic or other probabilistic methods to quantify the meaningfulness of each variant.

The method 200 may subsequently assign the calculated chi-square statistic as the frequency-score for each variant. Alternatively, the method 200 may assign a different value as the frequency-score. For example, if a variant is determined to be statistically significant, then the method 200 may assign a maximum frequency-score to the variant (e.g., 1). Conversely, if a variant is determined to be not statistically significant, then the method 200 may assign a minimum frequency-score to the variant (e.g., 0). In general, the frequency-score may be based on any calculated quantitative value, in which the higher the frequency-score, the more likely that the variant is a meaningful variant, for instance.

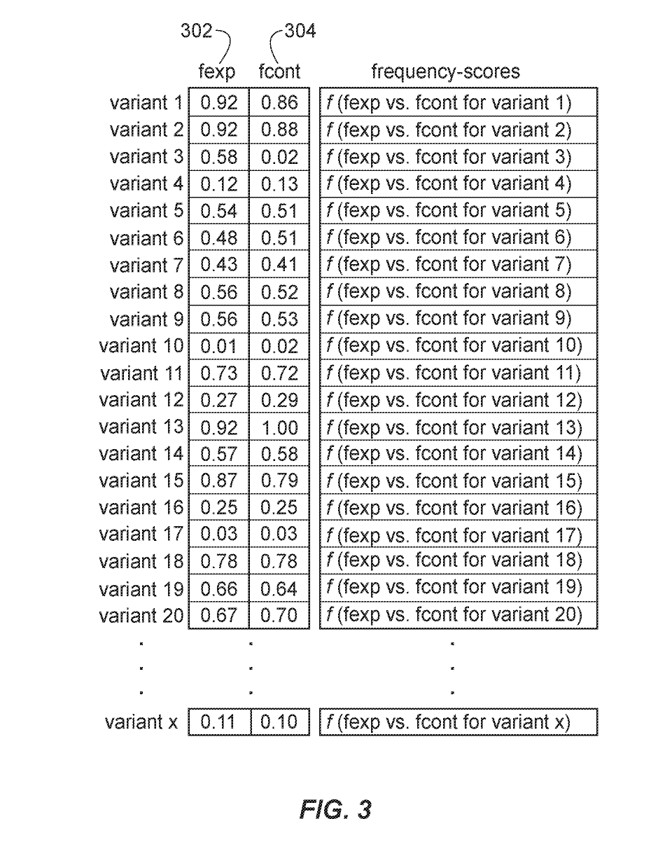

To illustrate the application of the process steps in blocks 204 and 206, consider FIG. 3, which depicts the variant frequency normalization of an example experimental dataset having variants 1 to x. The frequency at which each variant appears in the example experimental dataset is tabulated in column 302, while the frequency at which each variant appears in a corresponding example control dataset is tabulated in column 304. Qualitatively speaking, by examining the columns 302 and 304, the relative importance of each variant in the example experimental dataset can be determined. For example, the frequency at which most of the variants appear in the example experiment dataset is similar to the frequency at which most of the variants appear in the corresponding example control dataset. Thus, most of the variants in FIG. 3 are unlikely to be meaningful to the disease or biological phenomenon being investigated. However, the frequency of variant 3 in the example experimental dataset is much greater than the frequency of variant 3 in the corresponding example control dataset. Thus, variant 3 is very likely to be a meaningful variant in the example experimental dataset in this context. The data in the columns 302 and 304 can be further assessed quantitatively by calculating the frequency-score for each variant using, for example, the chi-square statistic.

Returning to FIG. 2, the method 200 proceeds to perform universal pairwise variant comparisons on the experimental dataset to determine the extent of biological inter-relatedness among all variants in the experimental dataset. To begin with, the method 200 performs pairwise comparisons between each variant in the experimental dataset (block 208). That is, each variant in the experimental dataset is compared against every other variant in the experimental dataset. Universal pairwise variant comparisons may also be applied to experimental and/or control datasets including positive and/or negative control datasets and may be applied using only a portion of the entire dataset(s) such as after data filtering according to desired biological properties of selected variants.

When compared in a pairwise fashion, most variants are irrelevant or have no relationship to one another in terms of their underlying biology. However, a handful of variants will have some type of relationship in connection with other variants within the experimental dataset. The types of relationships that any two given variants may have can be classified into two categories: intrinsic and extrinsic. In the intrinsic category, the relationships may identify whether two variants are (i) identical (or otherwise at the same genomic position on the same chromosome); (ii) in identical domains (e.g., both variants affect amino acid residues in close linear proximity), or (iii) in identical genes (e.g., both variants affect the same gene but are not closer than expected by chance). Importantly, these intrinsic relationships may be evaluated based on information in the experimental dataset alone and without the use of any supporting external databases. In the extrinsic category, the relationships may identify whether two variants are (i) within the same functional pathways (e.g., both variants affect genes that act in one or more functional pathways as defined by data on gene ontology or other empirical biological data); (ii) within the same gene family (e.g., both variants affect genes in a gene family based on nucleic acid sequence homology); (iii) in direct or indirect interactions with the same genes (e.g., both variants affect genes that interact together physically based on empirical biochemical data); or (iv) have similar gene expression profiles (e.g., both variants affect genes whose expression patterns in tissues is similar). These extrinsic relationships must be evaluated using data obtained from supporting external databases (e.g., the external databases 124 in FIG. 1).

The relationships identified for each pairwise variant comparison in the experimental dataset provide a type of qualitative measure. In order to quantify the relationships, the method 200 calculates and assigns a quantitative relatedness-score for each pairwise variant comparison in the experimental dataset (block 210). Generally speaking, the method 200 may use any mathematical or statistical methods to calculate and assign the relatedness-score. For example, a pairwise variant comparison may identify two variants that are in the same gene but are not identical. In this scenario, the method 200 may quantify this relationship by calculating and assigning a relatedness-score according to how biologically near or distant the two variants are to or from one another. As such, the pairwise comparison may be given a higher relatedness-score if the two variants are found to be closer together than if the two variants are farther apart. As another example, for a pairwise variant comparison in which the relationship between two variants is shown to be identical, the method 200 may calculate and assign a maximum relatedness-score (e.g., 1). Similarly, for a pairwise variant comparison that does not show any evidence of relationship between two variants, the method 200 may calculate and assign a minimum relatedness-score (e.g., 0). As a further example, for pairwise variant comparisons that show extrinsic relationships, the method 200 may assign relatedness-scores according to some predetermined values. The predetermined values may be calculated based on a 2×2 matrix of gene-to-gene comparisons compiled using data obtained from internal or external databases. In assigning relatedness-scores, the method 200 may first reference the 2×2 matrix to determine in which two genes the two variants from the pairwise comparisons are located, and then assign the corresponding predetermined values to the pairwise comparisons.

In some embodiments, a biological relatedness rule set may be applied to determine the quantitative relatedness-score for each pairwise comparison of variants. Such rule set may include rules that determine a type of intrinsic relationship between the variants in a pairwise comparison by comparing genome sequences of the variants, then assigning a quantitative score to the pair that indicates the type of relationship. To evaluate an extrinsic relationship between a pair of variants, the rule set may further include rules that access one or more external databases to access extrinsic data regarding the variants, then compare such extrinsic data to determine a type of extrinsic relationship between the pair of variants and assign a corresponding quantitative score that indicates the type of the extrinsic relationship. The extrinsic data obtained from external databases may include information regarding functional pathways associated with variants, gene families associated with variants, direct or indirect gene interactions associated with variants, or gene expression profiles associated with variants. The rule set may further include a rule to determine that a pair of variants is unrelated if no intrinsic or extrinsic relationship between the variants is identified by the other rules in the rule set.

Thus, the types of relationships that any two given variants may have can be classified by the rule set into two categories: intrinsic and extrinsic. In the intrinsic category, the relationships may identify whether two variants are (i) identical (or otherwise at the same genomic position on the same chromosome) calculable by a computer program using database lookups and objective data comparison; (ii) in identical domains (e.g., both variants affect amino acid residues in close linear proximity) calculable by a computer program using database lookups of known empirically determined protein domains and classification of which domain or domains are affected; or (iii) in identical genes (e.g., both variants affect the same gene but are not closer than expected by chance) determined by a computer program using custom genome annotation software with inputs from the human genome project data that defines the boundaries of all genes comprising the human genome. In the extrinsic category, the relationships may be quantitated using a computer program after consultation of existing databases of empirically determined biological information to determine whether two variants are (i) within the same functional pathways (e.g., both variants affect genes that act in one or more functional pathways as defined by data on gene ontology or other empirical biological data); (ii) within the same gene family (e.g., both variants affect genes in a gene family based on nucleic acid sequence homology); (iii) in direct or indirect interactions with the same genes (e.g., both variants affect genes that interact together physically based on empirical biochemical data); or (iv) have similar gene expression profiles (e.g., both variants affect genes whose expression patterns in tissues is similar). These extrinsic relationships must be evaluated using data obtained from supporting external databases (e.g., the external databases 124 in FIG. 1) and the results from each set of databases queries integrated by a computer program.

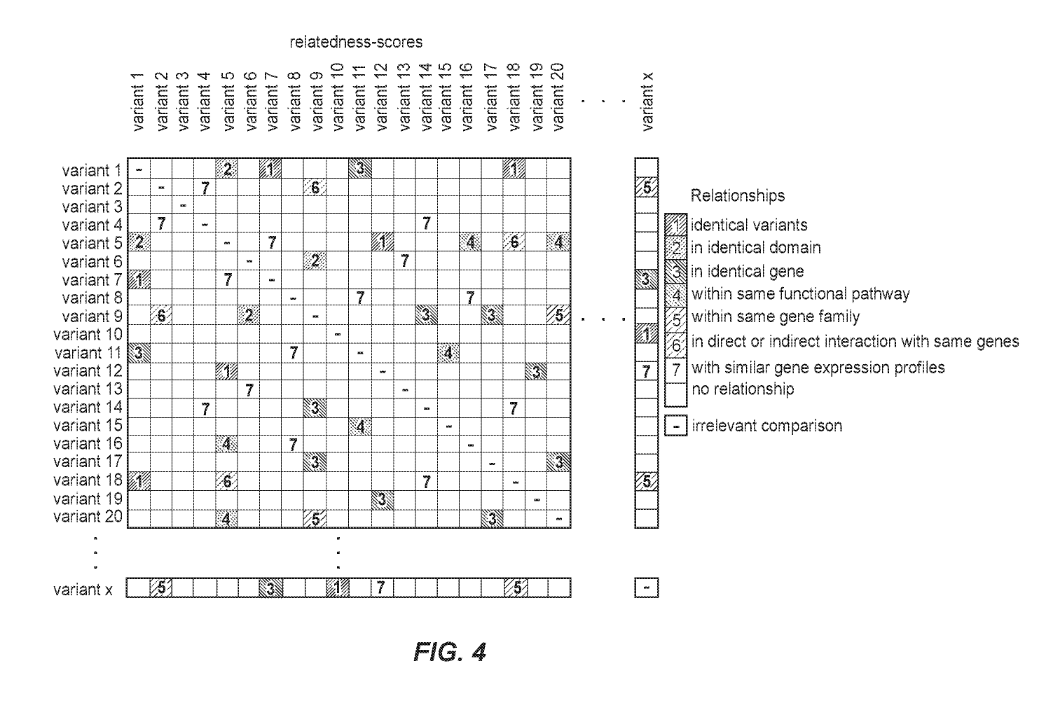

To illustrate the application of the process steps in blocks 208 and 210, consider FIG. 4, which depicts the pairwise comparison results for the example experimental dataset of FIG. 3. The results are tabulated in a 2×2 matrix of pairwise variant comparisons with the type of relationship for each pairwise comparison being indicated by numbers 1-7. The numbers 1-3 indicate intrinsic relationships, while the numbers 4-7 indicate extrinsic relationships. As can be seen in FIG. 4, most pairwise variant comparisons have no relationships. However, many pairwise comparisons do yield meaningful relationships. For example, consider variant 7, a comparison of variant 7 versus variant 1 shows that the two variants are identical. Also, a comparison of variant 7 versus variant 5 shows that the two variants have similar gene expression profiles. In general, the identified relationships are qualitative measures, but these relationships can be further assessed quantitatively by calculating the relatedness-score for each of the identified relationships. Depending on the specific methods of the relatedness-score calculation, the numbers in this 2×2 matrix may represent distinct categories of values as shown or otherwise may take values along a continuum with an infinite number of possible quantitative values (e.g., 5.34) for each entry depending on the specifics of the relatedness-score calculation. Moreover, in some embodiments, higher priority relationships may alternatively be assigned higher numerical values for the relationship score. As an example, because the comparison between variant 7 and variant 1 shows that the two variants are identical, a maximum relatedness-score of one (1) may be calculated and assigned to that comparison. As an additional example, comparison of variants 1 and 11 demonstrate that they are in the same gene (relationship category 3); in other methods of the relatedness-score calculation, this value may be modified from a smaller or higher value depending on the intrinsic or extrinsic biological properties of the two variants involved in the relatedness-score calculation (e.g., gene size).

Returning again to FIG. 2, once the relatedness-score is determined for each pairwise variant comparison in the experimental dataset, the method 200 calculates and assigns a frequency-corrected relatedness-score to each pairwise variant comparison in the experimental dataset (block 212). To do so, the method 200 combines the relatedness-score of each pairwise variant comparison (as determined in block 210), with the corresponding frequency-score of each variant in the pairwise comparison (as determined in block 206). In particular, the method 200 multiplies the frequency-score associated with each variant in the pairwise comparison with the relatedness-score of the pairwise comparison. By assigning the frequency-corrected relatedness-score to each pairwise variant comparison, the method 200 can further quantify the overall relevance of each pairwise variant comparison in the context of the entire experimental dataset.

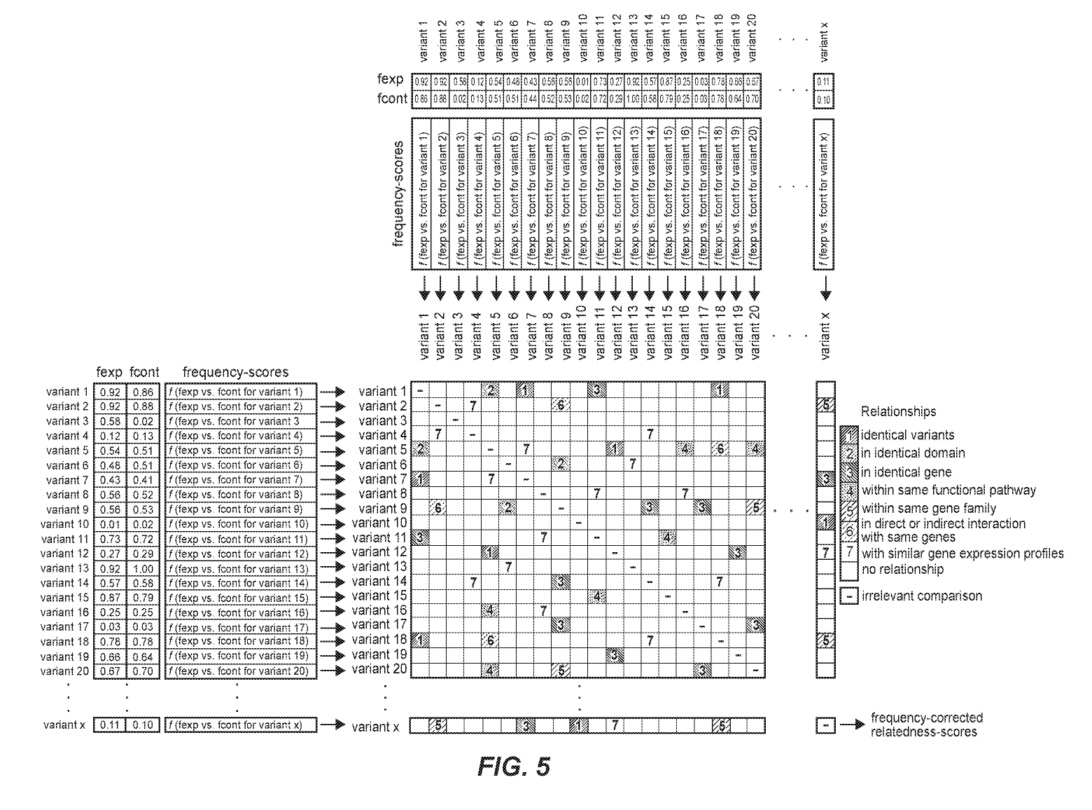

The application of the process steps in block 212 is illustrated in FIG. 5, which depicts the process of determining the frequency-corrected relatedness-scores for the pairwise comparison results of FIG. 4. In FIG. 5, the frequency-scores for the variants 1 to x (as shown in FIG. 3) are applied (e.g., multiplied) to the 2×2 matrix of pairwise comparisons to generate the frequency-corrected relatedness-scores.

With continued reference to FIG. 2, the method 200 may process the control dataset in a similar fashion as the experimental dataset. First, the method 200 performs variant frequency normalization on the control dataset. The method 200 determines the prevalence or frequency at which each variant in the control dataset appears in the experimental dataset and the control dataset (block 214). Again, this is a qualitative measure that may identify distinct subpopulations of variants that are more likely to be important than others in the control dataset.

To quantify the relative importance of each variant in the control dataset, the method 200 calculates and assigns a control-frequency-score for each variant in the control dataset (block 216). Similar to block 206, the method 200 may calculate and assign the control-frequency-score based on the chi-square statistic, for example.

Next, the method 200 performs universal pairwise variant comparisons on the control dataset. To start, the method 200 performs pairwise comparisons between each variant in the experimental dataset and each variant in control dataset (block 218). In other words, each variant in the experimental dataset is compared against each variant in the control dataset. Similar calculations can be performed using one or multiple control datasets depending on the nature of the experimental dataset. Moreover, in some embodiments only a subset of experimental data may be subjected to calculation of the values resulting from universal pairwise comparisons using control datasets. For example, in the case of a single genome sample comprising the experimental dataset, one control dataset may represent data derived from a healthy population unaffected by disease or not possessing a given biological trait, whereas a separate control dataset may represent data derived from a population of individuals affected by disease or otherwise possessing a certain biological trait.

To quantify the pairwise variant comparisons determined in block 218, the method 200 calculates and assigns a control-relatedness-score for each pairwise comparison between each variant in the experimental dataset and each variant in control dataset (block 220). The method 200 may determine the control-relatedness-score in a similar manner as the relatedness-score in block 210.

The method 200 also calculates and assigns a control-frequency-corrected relatedness-score to each pairwise comparison between each variant in the experimental dataset and each variant in the control dataset (block 222). To do so, the method 200 combines the control-relatedness-score of each pairwise variant comparison (as determined in block 220) with the corresponding frequency-score (as determined in block 206) and control-frequency-score (as determined in block 216) of the variants in the pairwise comparison. More particularly, the method 200 multiplies the frequency-score or the control-frequency-score associated with each variant in the pairwise comparison with the control-relatedness-score of the pairwise comparison.

Using the control-frequency-corrected relatedness-scores determined in block 222, the method 200 may proceed to calculate and assign a control-frequency-adjusted relatedness-score for each variant in the experimental dataset (block 224). More specifically, in block 218, each given variant in the experimental dataset was compared to each variant in the control dataset. As a result, pairwise comparisons exist between each given variant in the experimental dataset and each variant in the control dataset. Each of these pairwise comparisons associated with each given variant in the experimental dataset was then assigned a control-frequency-corrected relatedness-score in block 222. Now, by combining (e.g., summing) the corresponding control-frequency-corrected relatedness-scores for all the pairwise comparisons associated with each given variant in the experimental dataset, the method 200 can determine the control-frequency-adjusted relatedness-score for each given variant in the experimental dataset.

After determining the control-frequency-adjusted relatedness-score for each variant in the experimental dataset, the method 200 calculates and assigns a normalized frequency-corrected relatedness-score for each pairwise variant comparison in the experimental dataset (block 226). To accomplish this normalization, for each given variant in the experimental dataset, the method 200 divides the corresponding frequency-corrected relatedness-scores for all the pairwise comparisons associated with each given variant in the experimental dataset (as determined in block 212) by the control-frequency-adjusted relatedness-score for each given variant in the experimental dataset (as determined in block 224). The purpose of normalization is to eliminate artifacts caused by large biological interactomes or otherwise large or polymorphic genes. This information is essential to uncover the cause of diseases whose underlying genetic etiology is multi-factorial. Accordingly, the use of normalization serves to further highlight only those variants in experimental dataset that have high likelihoods to be meaningful variants.

Finally, the method 200 calculates and assigns a priority-score for each variant in the experimental dataset (block 228). For each given variant in the experimental dataset, the method 200 determines the priority-score by combining (e.g. summing) the corresponding normalized frequency-corrected relatedness-scores for all the pairwise comparisons associated with each given variant in the experimental dataset. The priority-score serves to rank each variant in the experimental dataset in terms of pathogenic importance. The priority-score will be low for variants in the experimental dataset that are common and/or have few similar variants within the experimental dataset as compared to the number of similar variants within the control dataset. By contrast, the priority-score will be high for less common or previously unreported variants with numerous similar variants within the experimental dataset but without multiple similar variants in the control dataset. In some embodiments, the method 200 may perform similar calculations to have the priority-score be minimized for important variants and maximized for unimportant variants.

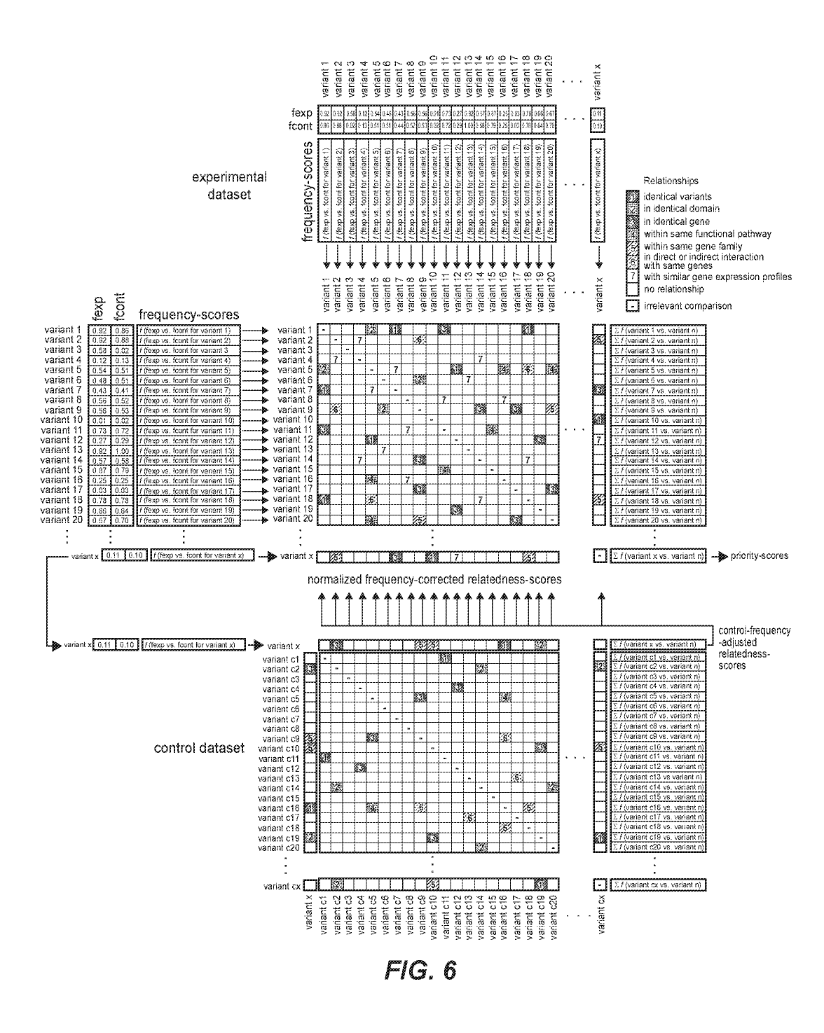

The application of the process steps in blocks 204-228 are summarized in FIG. 6, which depicts the process of determining the priority-score for each variant in the example experimental dataset of FIG. 3. As shown in FIG. 6, the corresponding example control dataset for the example experimental dataset of FIG. 3 is processed to determine the control-frequency-adjusted relatedness-score for each variant in the example experimental dataset. The control-frequency-adjusted relatedness-scores are then applied to the pairwise comparison results of the example experimental dataset (as shown in FIG. 5) to generate the normalized frequency-corrected relatedness-scores. Subsequently, the normalized frequency-corrected relatedness-scores are combined (e.g., summed) to produce the priority-score for each variant in the example experimental dataset.

Referring once more to FIG. 2, after the overall significance or rank of each variant in the experimental dataset is determined by calculation of the priority-score or components of the priority-score, the method 200 may generate visualizations of the variant ranking and potential for importance (block 230). The method 200 may then display the visualizations to the user (e.g., via the computing device 102 in FIG. 1).

Generally speaking, the method 200 may generate and display the visualizations to the user according to any desired format. In an example embodiment, the method 200 may organize the resultant data into clusters according to biologically meaningful information pertaining to one or more variants. For example, this process may first identify the variant with the highest priority-score which serves as an index variant for the first cluster. Next, the variant with the highest normalized frequency-corrected relatedness-score as determined from a variant to variant comparison with the index variant forms the first satellite variant. Subsequently, the variant with the next highest normalized frequency-corrected relatedness-score forms the second satellite variant. The process continues until there are no more variants that have non-zero normalized frequency-corrected relatedness-scores with the index variant. The index variant and all satellite variants that comprise the first cluster are removed from consideration in subsequent iterations of cluster formation. As such, the variant with the highest priority-score that was not included in the first cluster then forms the index variant for the second cluster. The variant with the highest normalized frequency-corrected relatedness-score as determined from a variant to variant comparison with the second index variant forms the first satellite variant for the second cluster. Multiple related clusters of variants may be produced in this manner until all variants have been organized into clusters. In essence, the variants that are most likely to be of relevance to the disease being studied are given greatest prominence with similar variants in close proximity within a distinct cluster.

These organized data clusters can be displayed to the user in any one of a variety of data visualization modes. For example, the data clusters may be presented with individual variants displayed in tables, cartograms, node-link diagrams, force-directed layouts, matrix views, etc. As another example, the data clusters may be presented in interactive graphical forms with variant importance being represented by icon size and inter-relatedness being represented by icon proximity. Other biologically relevant information can be depicted visually by assigning characteristics of icons representing individual variants or groups of variants (e.g., icon color). Hyperlinks may also be used to connect each variant or cluster with useful biological information in internal or external databases. Alternatively or additionally, information in the data clusters may be displayed according to user preference (e.g., organized as gene vs. variant, gene vs. sample or genome, variant vs. sample or genome, etc.).

To better demonstrate the mechanics of the process steps involved in the method 200, an example calculation is provided below. Consider, for example, an experimental dataset with four variants (V₁, V₂, V₃, V₄), and a control dataset with four variants (Vc₁, Vc₂, Vc₃, Vc₄).

To process the experimental dataset, the first step is to calculate the frequency-scores for V₁, V₂, V₃ and V₄.

The second step is to perform pairwise comparisons between each variant in the experimental dataset. To illustrate, the pairwise variant comparisons between V₁ and each variant in the experimental dataset are determined to be: (V₁ vs. V₂), (V₁ vs. V₃), and (V₁ vs. V₄).

The third step is to calculate the relatedness-scores for all the pairwise comparisons between each variant in the experimental dataset. To illustrate, the relatedness-scores for the pairwise variant comparisons between V₁ and each variant in the experimental dataset are as follows:

A=f(V₁vs.V₂)

B=f(V₁vs.V₃)

C=f(V₁vs.V₄).

The fourth step is to calculate the frequency-corrected relatedness-scores for all the pairwise comparisons between each variant in the experimental dataset. To illustrate, the frequency-corrected relatedness-scores for the pairwise variant comparisons between V₁ and each variant in the experimental dataset may be calculated as:

A′=A*(frequency-score of V₁)*(frequency-score of V₂)

B′=B*(frequency-score of V₁)*(frequency-score of V₃)

C′=C*(frequency-score of V₁)*(frequency-score of V₄).

To process the control dataset, the first step is to calculate the control-frequency-scores for Vc₁, Vc₂, Vc₃ and Vc₄.

The second step is to perform pairwise comparisons between each variant in the experimental dataset and each variant in the control dataset. To illustrate, the pairwise variant comparisons between V₁ and each variant in the control dataset are determined to be: (V₁ vs. Vc₁), (V₁ vs. Vc₂), (V₁ vs. Vc₃), and (V₁ vs. Vc₄). This step and the steps described below are repeated for V₂, V₃ and V₄.

The third step is to calculate the control-relatedness-scores for all the pairwise comparisons between each variant in the experimental dataset and each variant in the control dataset. To illustrate, the control-relatedness-scores for the pairwise variant comparisons between V₁ and each variant in the control dataset are as follows:

Wc=f(V₁vs.Vc₁)

Xc=f(V₁vs.Vc₂)

Yc=f(V₁vs.Vc₃)

Zc=f(V₁vs.Vc₄).

The fourth step is to calculate the control-frequency-corrected relatedness-scores for all the pairwise comparisons between each variant in the experimental dataset and each variant in the control dataset. To illustrate, the control-frequency-corrected relatedness-score for the pairwise comparisons between V₁ and each variant in the control dataset may be calculated as:

Wc′=Wc*(frequency-score of V₁)*(control-frequency-score of Vc₁)

Xc′=Xc*(frequency-score of V₁)*(control-frequency-score of Vc₂)

Yc′=Yc*(frequency-score of V₁)*(control-frequency-score of Vc₃)

Zc′=Zc*(frequency-score of V₁)*(control-frequency-score of Vc₄).

The fifth step is to calculate the control-frequency-adjusted relatedness-score for each variant in the experimental dataset. To illustrate, the control-frequency-adjusted relatedness-score for V₁ is calculated as: Wc′+Xc′+Yc′+Zc′.

Next, the normalized frequency-corrected relatedness-scores are calculated for all the pairwise comparisons between each variant in the experimental dataset. To illustrate, the normalized frequency-corrected relatedness-scores for the pairwise variant comparisons between V₁ and each variant in the experimental dataset are calculated as:

(V₁vs.V₂):(A′)/(Wc′+Xc′+Yc′+Zc′)

(V₁vs.V₃):(B′)/(Wc′+Xc′+Yc′+Zc′)

(V₁vs.V₄):(C′)/(Wc′+Xc′+Yc′+Zc′).

Finally, the priority-score is calculated for each variant in the experimental dataset. To illustrate, the priority-score for V₁ is calculated to be: (A′+B′+C′)/(Wc′+Xc′+Yc′+Zc′).

An aspect of the described systems and methods includes a computer-implemented method for grouping and visualizing genomic variants, the method comprising: receiving, via one or more processors, a set of genomic variants, wherein each of the genomic variants in the set includes a priority-score and a normalized frequency-corrected relatedness-score; forming, via one or more processors, one or more variant clusters by determining one or more index variants, wherein the one or more index variants are determined based on the priority-score of the each of the genomic variants in the set; determining, via one or more processors, one or more satellite variants for each of the one or more variant clusters based on comparisons of each of the one or more index variants with the normalized frequency-corrected relatedness-score of each of the genomic variants in the set; and displaying, via one or more processors, individual variants in each of the determined one or more variant clusters using icons of different characteristics such color, size or shape.

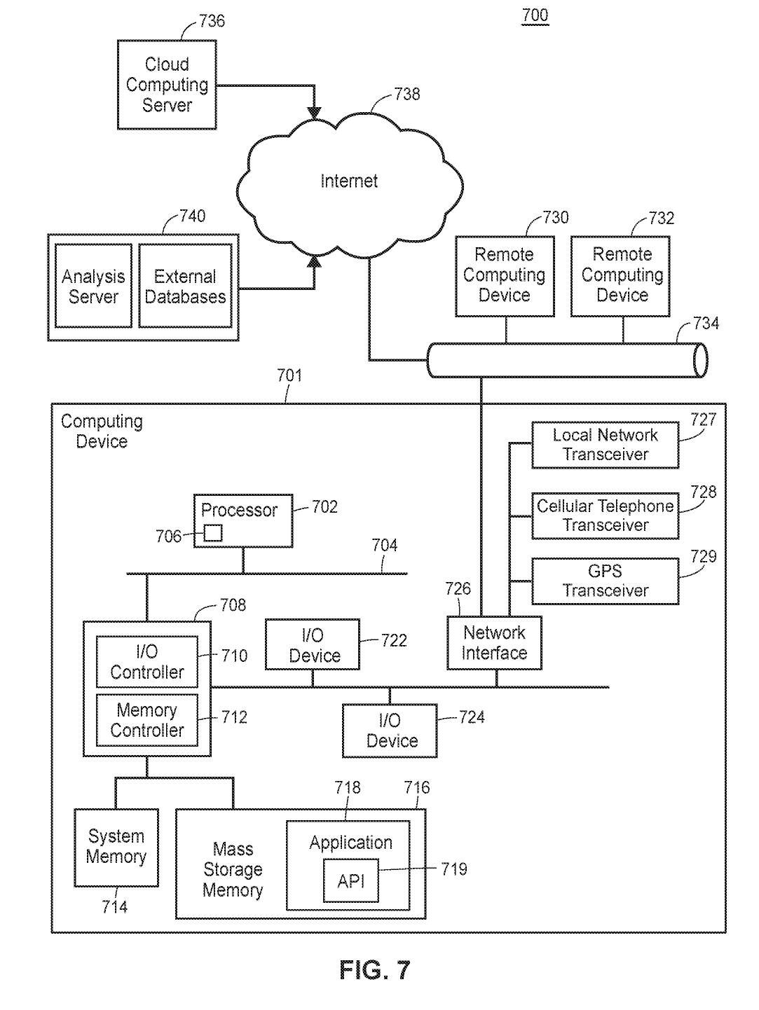

FIG. 7 is a block diagram of an example computing environment for an analysis system 700 having a computing device 701 that may be used to implement the systems and methods described herein. The computing device 701 may include one or more devices 102, a server 104, a mobile computing device (e.g., cellular phone, a tablet computer, a Wi-Fi-enabled device or other personal computing device capable of wireless or wired communication), a thin client, or other known type of computing device. As will be recognized by one skilled in the art, in light of the disclosure and teachings herein, other types of computing devices can be used that have different architectures. Processor systems similar or identical to the example analysis system 700 may be used to implement and execute the example system of FIG. 1, the method of FIG. 2, and the like. Although the example analysis system 700 is described below as including a plurality of peripherals, interfaces, chips, memories, etc., one or more of those elements may be omitted from other example processor systems used to implement and execute the example system 100. Also, other components may be added.

As shown in FIG. 7, the computing device 701 includes a processor 702 that is coupled to an interconnection bus 704. The processor 702 includes a register set or register space 706, which is depicted in FIG. 7 as being entirely on-chip, but which could alternatively be located entirely or partially off-chip and directly coupled to the processor 702 via dedicated electrical connections and/or via the interconnection bus 704. The processor 702 may be any suitable processor, processing unit or microprocessor. Although not shown in FIG. 7, the computing device 701 may be a multi-processor device and, thus, may include one or more additional processors that are identical or similar to the processor 702 and that are communicatively coupled to the interconnection bus 704.

The processor 702 of FIG. 7 is coupled to a chipset 708, which includes a memory controller 710 and a peripheral input/output (I/O) controller 712. As is well known, a chipset typically provides I/O and memory management functions as well as a plurality of general purpose and/or special purpose registers, timers, etc. that are accessible or used by one or more processors coupled to the chipset 708. The memory controller 710 performs functions that enable the processor 702 (or processors if there are multiple processors) to access a system memory 714 and a mass storage memory 716, that may include either or both of an in-memory cache (e.g., a cache within the memory 714) or an on-disk cache (e.g., a cache within the mass storage memory 716).

The system memory 714 may include any desired type of volatile and/or non-volatile memory such as, for example, static random access memory (SRAM), dynamic random access memory (DRAM), flash memory, read-only memory (ROM), etc. The mass storage memory 716 may include any desired type of mass storage device. For example, if the computing device 701 is used to implement an application 718 having an API 719 (including functions and instructions as described by the method 200 of FIG. 2). The mass storage memory 716 may include a hard disk drive, an optical drive, a tape storage device, a solid-state memory (e.g., a flash memory, a RAM memory, etc.), a magnetic memory (e.g., a hard drive), or any other memory suitable for mass storage. As used herein, the terms module, block, function, operation, procedure, routine, step, and method refer to tangible computer program logic or tangible computer executable instructions that provide the specified functionality to the computing device 701 and the analysis system 700. Thus, a module, block, function, operation, procedure, routine, step, and method can be implemented in hardware, firmware, and/or software. In one embodiment, program modules and routines (e.g., the application 718, the API 719, etc.) are stored in mass storage memory 716, loaded into system memory 714, and executed by a processor 702 or can be provided from computer program products that are stored in tangible computer-readable storage mediums (e.g., RAM, hard disk, optical/magnetic media, etc.).

The peripheral I/O controller 710 performs functions that enable the processor 702 to communicate with peripheral input/output (I/O) devices 722 and 724, a network interface 726, a local network transceiver 727, a cellular network transceiver 728, and a GPS transceiver 729 via the network interface 726. The I/O devices 722 and 724 may be any desired type of I/O device such as, for example, a keyboard, a display (e.g., a liquid crystal display (LCD), a cathode ray tube (CRT) display, etc.), a navigation device (e.g., a mouse, a trackball, a capacitive touch pad, a joystick, etc.), etc. The cellular telephone transceiver 728 may be resident with the local network transceiver 727. The local network transceiver 727 may include support for a Wi-Fi network, Bluetooth, Infrared, or other wireless data transmission protocols. In other embodiments, one element may simultaneously support each of the various wireless protocols employed by the computing device 701. For example, a software-defined radio may be able to support multiple protocols via downloadable instructions. In operation, the computing device 701 may be able to periodically poll for visible wireless network transmitters (both cellular and local network) on a periodic basis. Such polling may be possible even while normal wireless traffic is being supported on the computing device 701. The network interface 726 may be, for example, an Ethernet device, an asynchronous transfer mode (ATM) device, an 802.11 wireless interface device, a DSL modem, a cable modem, a cellular modem, etc., that enables the system 100 to communicate with another computer system having at least the elements described in relation to the system 100.

While the memory controller 712 and the I/O controller 710 are depicted in FIG. 7 as separate functional blocks within the chipset 708, the functions performed by these blocks may be integrated within a single integrated circuit or may be implemented using two or more separate integrated circuits. The analysis system 700 may also implement the application 718 on remote computing devices 730 and 732. The remote computing devices 730 and 732 may communicate with the computing device 701 over an Ethernet link 734. In some embodiments, the application 718 may be retrieved by the computing device 701 from a cloud computing server 736 via the Internet 738. When using the cloud computing server 736, the retrieved application 718 may be programmatically linked with the computing device 701. The application 718 may be a Java® applet executing within a Java® Virtual Machine (JVM) environment resident in the computing device 701 or the remote computing devices 730, 732. The application 718 may also be “plug-ins” adapted to execute in a web-browser located on the computing devices 701, 730, and 732. In some embodiments, the application 718 may communicate with backend components 740 such as the analysis server 104 and the external databases 124 via the Internet 738.

The system 700 may include but is not limited to any combination of a LAN, a MAN, a WAN, a mobile, a wired or wireless network, a private network, or a virtual private network. Moreover, while only two remote computing devices 730 and 732 are illustrated in FIG. 7 to simplify and clarify the description, it is understood that any number of client computers are supported and can be in communication within the system 700.

Additionally, certain embodiments are described herein as including logic or a number of components, modules, or mechanisms. Modules may constitute either software modules (e.g., code or instructions embodied on a machine-readable medium or in a transmission signal, wherein the code is executed by a processor) or hardware modules. A hardware module is tangible unit capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. As used herein, “hardware-implemented module” refers to a hardware module. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network (e.g., the Internet) and via one or more appropriate interfaces (e.g., application program interfaces (APIs)).

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Some portions of this specification are presented in terms of algorithms or symbolic representations of operations on data stored as bits or binary digital signals within a machine memory (e.g., a computer memory). These algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “some embodiments” or “an embodiment” means that a particular element, feature, structure, or characteristic described in connection with the embodiment is included in at least one embodiment. The appearances of the phrase “in some embodiments” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

Further, the figures depict preferred embodiments of a system and method for automatically identifying and prioritizing genomic variants of pathogenic importance from genome sequence datasets for purposes of illustration only. One skilled in the art will readily recognize from the following discussion that alternative embodiments of the structures and methods illustrated herein may be employed without departing from the principles described herein.

Upon reading this disclosure, those of skill in the art will appreciate still additional alternative structural and functional designs for a system and a method for automatically identifying and prioritizing genomic variants of pathogenic importance from genome sequence datasets through the disclosed principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims. Such modifications, changes and variations will be useful in interpreting data associated with a single individual as well as data associated with multiple individuals as well as multiple sets of data associated with multiple individuals. These modifications, changes and variations may also be applied to analysis of data from one or more of any species of organism including but not limited to humans, other mammalian species, other non-mammalian animal species and any other organisms including but not limited to plant species, bacterial species and viral species. These modifications, changes and variations will be useful in applying the method to interpretation and analysis of genome sequencing data from DNA samples including but not limited to tumor and matched constitutional normal samples, father-mother-child trios involving a proband with a presumed constitutional or other genetic disorder, members of entire family pedigrees or multiple complete or partial family pedigrees, or entire groups of individuals with a common disease process or biological phenomenon or phenotype. These modifications, changes and variations will also be useful in addressing specific questions pertaining to any phenomenon with a genetically determined component including but not limited to disease-risk prediction, predicted response to specific medications, likelihood of development of various physical and behavioral traits, likelihood of producing offspring with various genetically determined characteristics, likelihood of an individual or group of individuals to be of a certain ethnicity, likelihood that an individual or group of individuals shares an ancestor in common with another individual or another group of individuals, likelihood that two datasets of genome sequencing data were derived from the same or related individuals, etc. Moreover, these modification, changes and variations will be useful in applying the method to sequencing data that results from analysis of biomolecules other than genomic DNA itself, such as RNA. Further, these modifications, changes and variations will be useful in identifying patterns in data derived from modified DNA genome sequencing experiments such as those used to determine genomic regions influenced by any of a number of epigenetic modifications including but not limited to DNA methylation, histone modification, and other epigenetic modifications mediated by DNA-protein interactions. Additionally, these modifications, changes and variations will be useful in permitting the understanding of output generated using the modified method by those outside of the medical field or otherwise with limited biological background.

What is claimed is:

1. A computer-implemented method for automatically identifying and prioritizing variants in a dataset, the method comprising:
accessing, by one or more processors, the dataset, wherein the dataset includes genomic sequence data of a target dataset and a control dataset;
calculating, by the one or more processors, a frequency-score for each variant in the target dataset, wherein the frequency-score is based upon statistical frequencies with which the respective variant appears in each of the target dataset and the control dataset;
for each pair of variants in the target dataset:
performing, by the one or more processors, pairwise comparison between the respective variants of the pair;
calculating, by the one or more processors, a relatedness-score for the pair based upon the pairwise comparison; and
calculating, by the one or more processors, a frequency-corrected relatedness-score for the pair based upon the relatedness-score of the pair and the frequency scores of the respective variants;
calculating, by the one or more processors, a control-frequency-score for each variant in the control dataset, wherein the control-frequency-score is based upon statistical frequencies with which the respective variant appears in each of the target dataset and the control dataset;
for each control pair of (i) a target variant in the target dataset and (ii) a control variant in the target dataset:
performing, by the one or more processors, pairwise comparison between the target and control variants of the control pair;
calculating, by the one or more processors, a control-relatedness-score for the control pair based upon the pairwise comparison; and
calculating, by the one or more processors, a control-frequency-corrected relatedness-score for the control pair based upon the control-relatedness-score of the control pair, the frequency score of the target variant, and the control-frequency score of the control variant;
calculating, by the one or more processors, a control-frequency-adjusted relatedness-score for each variant in the target dataset, wherein the control-frequency-adjusted relatedness-score for each respective variant is based upon the control-frequency-corrected relatedness-scores of the control pairs in which the respective variant is included in the control pair as the target variant;
calculating, by the one or more processors, a normalized frequency-corrected relatedness-score for each pair of variants in the target dataset, wherein the normalized frequency-corrected relatedness-score is associated with one of the variants of the pair and is based upon (i) the frequency-corrected relatedness-score of the pair and (ii) the control-frequency-adjusted relatedness-scores of the one of the variants of the pair; and
calculating, by the one or more processors, a priority-score for each variant in the target dataset, wherein the priority-score of each respective variant is based upon the normalized frequency-corrected relatedness-scores associated with the respective variant.

2. The computer-implemented method of claim 1, wherein:
calculating the frequency-score for each variant in the target dataset includes: (i) calculating a first frequency of the respective variant in the target dataset, (ii) calculating a second frequency of the respective variant in the control dataset, and (iii) calculating the frequency-score based upon a difference between the first frequency and the second frequency; and
calculating the control-frequency-score for each variant in the control dataset includes: (i) calculating a first control-frequency of the respective variant in the target dataset, (ii) calculating a second control-frequency of the respective variant in the control dataset, and (iii) calculating the control-frequency-score based upon a difference between the first control-frequency and the second control-frequency.

3. The computer-implemented method of claim 1, wherein:
calculating the control-frequency-adjusted relatedness-score for each variant in the target dataset includes summing all the control-frequency-corrected relatedness-scores of the control pairs for which the respective variant is the target variant; and
calculating the normalized frequency-corrected relatedness-score for each pair of variants in the target dataset includes dividing the frequency-corrected relatedness-score of the pair by the control-frequency-adjusted relatedness-score of the one of the variants of the pair.

4. The computer-implemented method of claim 1, wherein calculating the priority-score for each variant includes summing all the normalized frequency-corrected relatedness-scores associated with the respective variant.

5. The computer-implemented method of claim 1, wherein the priority-score of each variant indicates a likelihood that the respective variant contributes to a disease process.

6. The computer-implemented method of claim 1, wherein the one or more processors are disposed in a plurality of servers and perform at least a portion of the pairwise comparisons by parallel computing in the plurality of servers.

7. The computer-implemented method of claim 1, wherein performing the pairwise comparison between each pair or control pair of variants includes applying a rule set to calculate a biological relationship between the respective variants, wherein the biological relationship comprises one of an intrinsic relationship identifying whether two variants are: (i) identical or otherwise at the same genomic position, (ii) in identical domain, or (iii) in identical gene, or an extrinsic relationship identifying whether two variants are: (i) within the same functional pathway, (ii) within the same gene family, (ii) in direct or indirect interaction with the same genes, or (iv) have similar gene expression profiles.

8. A non-transitory computer-readable medium storing computer-readable instructions for automatically identifying and prioritizing variants in a dataset that, when executed by one or more processors of a computer system, cause the computer system to:
access the dataset, wherein the dataset includes genomic sequence data of a target dataset and a control dataset;
calculate, a frequency-score for each variant in the target dataset, wherein the frequency-score is based upon statistical frequencies with which the respective variant appears in each of the target dataset and the control dataset;
for each pair of variants in the target dataset:
perform, pairwise comparison between the respective variants of the pair;
calculate a relatedness-score for the pair based upon the pairwise comparison; and
calculate a frequency-corrected relatedness-score for the pair based upon the relatedness-score of the pair and the frequency scores of the respective variants;
calculate a control-frequency-score for each variant in the control dataset, wherein the control-frequency-score is based upon statistical frequencies with which the respective variant appears in each of the target dataset and the control dataset;
for each control pair of (i) a target variant in the target dataset and (ii) a control variant in the target dataset:
perform pairwise comparison between the target and control variants of the control pair;
calculate a control-relatedness-score for the control pair based upon the pairwise comparison; and
calculate a control-frequency-corrected relatedness-score for the control pair based upon the control-relatedness-score of the control pair, the frequency score of the target variant, and the control-frequency score of the control variant;
calculate a control-frequency-adjusted relatedness-score for each variant in the target dataset, wherein the control-frequency-adjusted relatedness-score for each respective variant is based upon the control-frequency-corrected relatedness-scores of the control pairs in which the respective variant is included in the control pair as the target variant;
calculate, a normalized frequency-corrected relatedness-score for each pair of variants in the target dataset, wherein the normalized frequency-corrected relatedness-score is associated with one of the variants of the pair and is based upon (i) the frequency-corrected relatedness-score of the pair and (ii) the control-frequency-adjusted relatedness-score of the one of the variants of the pair; and
calculate a priority-score for each variant in the target dataset, wherein the priority-score of each respective variant is based upon the normalized frequency-corrected relatedness-scores associated with the respective variant.

9. The non-transitory computer-readable medium of claim 8, wherein:
the instructions that cause the computer system to calculate the frequency-score for each variant in the target dataset cause the computer system to: (i) calculate a first frequency of the respective variant in the target dataset, (ii) calculate a second frequency of the respective variant in the control dataset, and (iii) calculate the frequency-score based upon a difference between the first frequency and the second frequency; and
the instructions that cause the computer system to calculate the control-frequency-score for each variant in the control dataset cause the computer system to: (i) calculate a first control-frequency of the respective variant in the target dataset, (ii) calculate a second control-frequency of the respective variant in the control dataset, and (iii) calculate the control-frequency-score based upon a difference between the first control-frequency and the second control-frequency.

10. The non-transitory computer-readable medium of claim 8, wherein:
the instructions that cause the computer system to calculate the control-frequency-adjusted relatedness-score for each variant in the target dataset cause the computer system to sum all the control-frequency-corrected relatedness-scores of the control pairs for which the respective variant is the target variant; and
the instructions that cause the computer system to calculate the normalized frequency-corrected relatedness-score for each pair of variants in the target dataset cause the computer system to divide the frequency-corrected relatedness-score of the pair by the control-frequency-adjusted relatedness-score of the one of the variants of the pair.

11. The non-transitory computer-readable medium of claim 8, wherein the instructions that cause the computer system to calculate the priority-score for each variant cause the computer system to sum all the normalized frequency-corrected relatedness-scores associated with the respective variant.

12. The non-transitory computer-readable medium of claim 8, wherein the priority-score of each variant indicates a likelihood that the respective variant contributes to a disease process.

13. The non-transitory computer-readable medium of claim 8, wherein the instructions are configured to be executed by or more processors disposed in a plurality of servers and perform at least a portion of the pairwise comparisons by parallel computing in the plurality of servers.

14. The non-transitory computer-readable medium of claim 8, wherein the instructions that cause the computer system to perform the pairwise comparison between each pair or control pair of variants cause the computer system to apply a rule set to calculate a biological relationship between the respective variants, wherein the biological relationship comprises one of an intrinsic relationship identifying whether two variants are: (i) identical or otherwise at the same genomic position, (ii) in identical domain, or (iii) in identical gene, or an extrinsic relationship identifying whether two variants are: (i) within the same functional pathway, (ii) within the same gene family, (ii) in direct or indirect interaction with the same genes, or (iv) have similar gene expression profiles.

15. A computer system for automatically identifying and prioritizing variants in a dataset, the system comprising:
one or more dataset repositories storing the dataset, including genomic sequence data of a target dataset and a control dataset; and
one or more processors communicatively connected to the one or more dataset repositories; and
a memory communicatively connected to the one or more processors and storing instructions that, when executed by the one or more processors, cause the computer system to:
access the target dataset and the control dataset of the one or more dataset repositories;
calculate, a frequency-score for each variant in the target dataset, wherein the frequency-score is based upon statistical frequencies with which the respective variant appears in each of the target dataset and the control dataset;
for each pair of variants in the target dataset:
perform, pairwise comparison between the respective variants of the pair;
calculate a relatedness-score for the pair based upon the pairwise comparison; and
calculate a frequency-corrected relatedness-score for the pair based upon the relatedness-score of the pair and the frequency scores of the respective variants;
calculate a control-frequency-score for each variant in the control dataset, wherein the control-frequency-score is based upon statistical frequencies with which the respective variant appears in each of the target dataset and the control dataset;
for each control pair of (i) a target variant in the target dataset and (ii) a control variant in the target dataset:
perform pairwise comparison between the target and control variants of the control pair;
calculate a control-relatedness-score for the control pair based upon the pairwise comparison; and
calculate a control-frequency-corrected relatedness-score for the control pair based upon the control-relatedness-score of the control pair, the frequency score of the target variant, and the control-frequency score of the control variant;
calculate a control-frequency-adjusted relatedness-score for each variant in the target dataset, wherein the control-frequency-adjusted relatedness-score for each respective variant is based upon the control-frequency-corrected relatedness-scores of the control pairs in which the respective variant is included in the control pair as the target variant;
calculate, a normalized frequency-corrected relatedness-score for each pair of variants in the target dataset, wherein the normalized frequency-corrected relatedness-score is associated with one of the variants of the pair and is based upon (i) the frequency-corrected relatedness-score of the pair and (ii) the control-frequency-adjusted relatedness-score of the one of the variants of the pair; and
calculate a priority-score for each variant in the target dataset, wherein the priority-score of each respective variant is based upon the normalized frequency-corrected relatedness-scores associated with the respective variant.

16. The computer system of claim 15, wherein:
the instructions that cause the computer system to calculate the frequency-score for each variant in the target dataset cause the computer system to: (i) calculate a first frequency of the respective variant in the target dataset, (ii) calculate a second frequency of the respective variant in the control dataset, and (iii) calculate the frequency-score based upon a difference between the first frequency and the second frequency; and
the instructions that cause the computer system to calculate the control-frequency-score for each variant in the control dataset cause the computer system to: (i) calculate a first control-frequency of the respective variant in the target dataset, (ii) calculate a second control-frequency of the respective variant in the control dataset, and (iii) calculate the control-frequency-score based upon a difference between the first control-frequency and the second control-frequency.

17. The computer system of claim 15, wherein
the instructions that cause the computer system to calculate the control-frequency-adjusted relatedness-score for each variant in the target dataset cause the computer system to sum all the control-frequency-corrected relatedness-scores of the control pairs for which the respective variant is the target variant; and
the instructions that cause the computer system to calculate the normalized frequency-corrected relatedness-score for each pair of variants in the target dataset cause the computer system to divide the frequency-corrected relatedness-score of the pair by the control-frequency-adjusted relatedness-score of the one of the variants of the pair.

18. The computer system of claim 15, wherein the instructions that cause the computer system to calculate the priority-score for each variant cause the computer system to sum all the normalized frequency-corrected relatedness-scores associated with the respective variant.

19. The computer system of claim 15, wherein:
the one or more processors are disposed in a plurality of servers; and
the instructions cause the one or more processors to perform at least a portion of the pairwise comparisons by parallel computing in the plurality of servers.

20. The computer system of claim 15, wherein the instructions that cause the computer system to perform the pairwise comparison between each pair or control pair of variants cause the computer system to apply a rule set to calculate a biological relationship between the respective variants, wherein the biological relationship comprises one of an intrinsic relationship identifying whether two variants are: (i) identical or otherwise at the same genomic position, (ii) in identical domain, or (iii) in identical gene, or an extrinsic relationship identifying whether two variants are: (i) within the same functional pathway, (ii) within the same gene family, (ii) in direct or indirect interaction with the same genes, or (iv) have similar gene expression profiles.
